(19) United States
(12) Patent Application Publication
(10) Pub. No.: US 2025/0161298 A1
(43) Pub. Date: May 22, 2025

(54) METHODS OF TREATING, AMELIORATING, OR PREVENTING HEART FAILURE, AND METHODS OF PROMOTING HEART MUSCLE GROWTH

(71) Applicant: Yale University, New Haven, CT (US)

(72) Inventors: Stuart Campbell, New Haven, CT (US); Shi Chen, New Haven, CT (US); Daniel Jacoby, New Haven, CT (US)

(21) Appl. No.: 18/840,040

(22) PCT Filed: Feb. 22, 2023
(86) PCT No.: PCT/US2023/062980

§ 371 Date: Aug. 20, 2024

Related U.S. Application Data
(60) Provisional application No. 63/312,590, filed on Feb. 22, 2022.

(51) Int. Cl. A61K 31/496; A61P 9/04

(52) U.S. Cl. CPC A61K 31/496; A61P 9/04

(57) ABSTRACT

Described herein is a method of treating, ameliorating and/or preventing heart failure in a subject in need thereof. The method includes administering to the subject an effective amount of a myotrope using a dosing scheme having an administration-withdrawal cycle. Also described herein are a method of promoting growth of a heart muscle, the method including intermittently contacting the heart muscle with a myotrope. Also described herein are kits for performing the methods described herein.

### CROSS-REFERENCE TO RELATED APPLICATIONS

The present application claims priority under 35 U.S.C. § 119 (e) to U.S. Provisional Patent Application No. 63/312,590, filed Feb. 22, 2022, which is incorporated herein by reference in its entirety.

### STATEMENT REGARDING FEDERALLY SPONSORED RESEARCH

This invention was made with government support under 1653160 awarded by the National Science Foundation. The government has certain rights in the invention.

### BACKGROUND

Heart failure (HF) is a clinical syndrome defined by an imbalance between cardiac function and metabolic demands of the body, and is a final pathway for many diseases that affect the heart. HF affects over 26 million people worldwide, with more than 3.5 million people newly diagnosed every year, and the prevalence increases with age. HF is typically further categorized as either “reduced” or “preserved” ejection fraction. The disease is most often caused by coronary artery disease; other etiologies include hypertensive heart disease, valvular heart disease and idiopathic causes. HF progresses through stages with compensatory mechanisms characterized by increased sympathetic tone, peripheral vasoconstriction, and activation of various neurohormonal pathways. These adaptive properties provide short-term relief but can be damaging with long-term or prolonged activation. Patients experience dyspnea, fatigue, and fluid retention and eventually develop pulmonary congestion and peripheral edema. Several pharmacological and nonpharmacological interventions have been shown to reduce the rate of HF hospitalizations and improve mortality, but mortality and morbidity still remain high.

HF is a leading cause of mortality in the US, and medical therapies for HF remain limited despite intensive efforts. Therefore, there is a need for novel methods of treating, ameliorating, and/or preventing HF. The present disclosure addresses this need.

### SUMMARY

In some aspects, the present study is directed to the following non-limiting embodiments.

Method of Treating, Ameliorating, and/or Preventing Heart Failure

In some aspects, the present invention is directed to a method of treating, ameliorating, and/or preventing heart failure in a subject in need thereof.

In some embodiments, the method includes administering to the subject an effective amount of a myotrope using a dosing scheme having an administration-withdrawal cycle.

In some embodiments, the dosing scheme includes administering the myotrope at a frequency of less than two doses per day.

In some embodiments, the plasma concentration of the myotrope in the subject cycles between above a first threshold concentration and below a second threshold concentration during the administration, and the first threshold concentration is equal to or higher than the second threshold concentration.

In some embodiments, the myotrope includes omecamtiv mecarbil (OM), APD418 (beta3-AR antagonist), danicamtiv, levosimendan, or combinations thereof.

In some embodiments, the method causes productive growth of a heart muscle in the subject.

In some embodiments, the subject is a mammal.

In some embodiments, the subject is a human.

### Method of Causing Productive Growth of a Heart Muscle

In some aspects, the present invention is directed to a method of causing productive growth of a heart muscle.

In some embodiments, the method includes intermittently contacting the heart muscle with a myotrope.

In some embodiments, the heart muscle is contacted with the myotrope at a frequency of less than once per day.

In some embodiments, the concentration of the myotrope being contacted with the heart muscle cycles between above a first threshold concentration and below a second threshold concentration during the contact-withdrawal cycle, and the first threshold concentration is equal to or higher than the second threshold concentration.

In some embodiments, the myotrope includes omecamtiv mecarbil (OM), APD418 (beta3-AR antagonist), danicamtiv, levosimendan, or combinations thereof.

In some embodiments, the heart muscle is in an engineered heart tissue.

In some embodiments, the heart muscle is in the heart of a subject.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1J demonstrate that long-term constant omecamtiv mecarbil (OM) dosing provides systolic improvement at significant diastolic costs, in accordance with some embodiments. FIG. 1A: Experimental design of 8-day constant OM dosing (OM-Ctrl, n=14) vs. constant DMSO of the same dose (vehicle, n=15), with data collected from two separate differentiation batches. FIG. 1B: Representative twitches at 1 Hz. FIG. 1C: Cross-sectional areas measured with optical coherence tomography (p=0.0019). FIG. 1D: Systolic peak force at 1 Hz (p=0.0076). FIG. 1E: Systolic peak stress at 1 Hz (p=0.0129). FIG. 1F: Time to peak (TTP) at 1 Hz (p<0.0001). FIG. 1G: Time to 50% relaxation (RT50) at 1 Hz (p<0.0001). FIG. 1H: Frank-Starling gain from −5 to 5% stretch (p<0.0001). FIG. 1I: Passive stress at 1 Hz from −5 to 5% stretch. FIG. 1J: Passive stiffness from −3 to 3% stretch. Unpaired two-tailed t tests (FIGS. 1C-1G) and two-way ANOVA with repeated measures (FIGS. 1H-1I) were performed. * p<0.05, ** p<0.005, **** p<0.0001.

FIGS. 2A-2J demonstrate that pulsed OM dosing provides systolic improvement over constant dosing, in accordance with some embodiments. FIG. 2A: Experimental design of 8-day constant OM dosing (Constant, n=10) vs. pulsed OM dosing (Pulse, n=11), with data collected from two separate differentiation batches. FIG. 2B: Representative twitches at 1 Hz. FIG. 2C: Cross-sectional areas measured with optical coherence tomography (p=0.0351). FIG. 2D: Systolic peak force at 1 Hz. FIG. 2E: Systolic peak stress at 1 Hz (p=0.0147). FIG. 2F: Time to peak (TTP) at 1 Hz. FIG. 2G: Time to 50% relaxation (RT50) at 1 Hz (p=0.0284). FIG. 2H: Frank-Starling gain from −5 to 5% stretch (p=0.003). FIG. 2I: Passive stress at 1 Hz from −5 to 5% stretch (p<0.0001). FIG. 2J: Passive stiffness from −3 to 3% stretch. Unpaired two-tailed t tests (FIGS. 2C-2G) and two-way ANOVA with repeated measures (FIGS. 2H-2I) were performed. * p<0.05, ** p<0.005, **** p<0.0001.

FIGS. 3A-3J demonstrate that pulsed OM dosing provides systolic improvement over constant dosing in a mutant dilated cardiomyopathy line E54K, in accordance with some embodiments. FIG. 3A: Experimental design of 8-day constant OM dosing with 24 hr washout (Constant, n=13), pulsed OM dosing with washout (Pulse, n=15), constant OM dosing without washout (n=12), and vehicle with washout (n=13), with data collected from two separate differentiation batches. FIG. 3B: Non-linear regressions of averaged TTP and RT50 for OM-Ctrl (n=4) to measure OM washout. FIG. 3C: Representative twitches at 1 Hz. FIG. 3D: Cross-sectional areas measured with optical coherence tomography (Constant vs. pulse p=0.0252, pulse vs. OM-Ctrl p=0.011). FIG. 3E: Systolic peak force at 1 Hz (Constant vs. OM-Ctrl p=0.0003, pulse vs. OM-Ctrl p=0.0103, vehicle vs. OM-Ctrl p=0.0005). FIG. 3F: Systolic peak stress at 1 Hz (Constant vs. pulse p=0.0014, Pulse vs. vehicle p=0.0046, constant vs. OM-Ctrl p=0.0013, vehicle vs. OM-Ctrl p=0.0039). FIG. 3G: Time to peak (TTP) at 1 Hz (Constant vs. OM-Ctrl p<0.0001, pulse vs. OM-Ctrl<0.0001, vehicle vs. OM-Ctrl p<0.0001, constant vs. vehicle p=0.0016, pulse vs. vehicle p=0.0016). FIG. 3H: Time to 50% relaxation (RT50) at 1 Hz (OM-Ctrl vs, all other groups p<0.0001). FIG. 3I: Frank-Starling gain from −5 to 5% stretch (OM-Ctrl vs. all other groups p<0.0001, constant vs. vehicle p<0.0001, pulse vs. vehicle p<0.0001). FIG. 3J: Passive stress at 1 Hz from −5 to 5% stretch (OM-Ctrl vs. all other groups p<0.0001, constant vs. pulse p<0.0001, constant vs. vehicle [<0.0001). One-way ANOVA with planned comparisons (FIGS. 3D-3H) and two-way ANOVA with repeated measurements (FIGS. 3I-3J) were performed. * p<0.05, ** p<0.01, **** p<0.0001.

FIGS. 4A-4G show the effects of chronic intermittent (pulsed) administration of 1.0 μM cardiac myosin-specific activator danicamtiv on engineered heart tissues (EHT) contractility. Behavior is compared against EHTs receiving constant administration of danicamtiv over the same 7-day period. Measured properties include the force (FIG. 4A), the peak twitch contraction stress (FIG. 4B), the time to peak twitch stress (TTP) (FIG. 4C), the time to 50% relaxation (RT50), the EHT cross-sectional area (CSA) (FIG. 4E), the length dependent activation (LDA) (FIG. 4F), and the diastolic stress-stretch relationship (FIG. 4G).

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Heart failure (HF) is a leading cause of mortality in the US. Medical therapies for heart failure remain limited despite intensive efforts. A new class of pharmaceuticals, so-called myotropes or cardiac sarcomere activators, have been developed for treating or preventing HF. Myotropes are intended to stimulate cardiac contraction, cause the heart to contract more forcefully, and thereby treat heart failure. Existing method of myotrope administration relies on maintaining a constant plasma concentration of the drug (see e.g., US 2020/0155547 A1). Omecamtiv mecarbil (OM) is the first myotrope to complete Phase 3 clinical trials. The clinical trial, however; did not meet the primary clinical end-point of reducing mortality in heart failure patients (Teerlink et al., N Engl J Med. 2021 Jan. 14; 384 (2): 105-116 and Teerlink et al, J Am Coll Cardiol. 2021 July, 78 (2) 97-108), and only severe heart failure group showed primary end point benefit (Felker et al., JAMA Cardiol. 2022 Jan. 1; 7 (1): 26-34).

The study described herein (“the present study”) describes the mixed effects of a non-limiting exemplary myotrope, omecamtiv mecarbil. Specifically, using engineered heart tissues (EHTs) as a non-limiting heart tissue model, the present study discovered that omecamtiv mecarbil makes heart tissue contract more forcefully. However, this agent also prevents the heart muscles from fully relaxing between beats. This lack of complete relaxation of the heart muscles impedes filling of the heart chambers with blood between beats and likely limits the efficacy of the drug.

Surprisingly, the present study further discovers that, after withdrawing heart tissue from OM treatment, the benefits of stronger contraction persisted for at least 24 hours without the negative side effects. Furthermore, the present study, in an exemplary in vitro study using human engineered heart tissues, found that an “intermittent” administration strategy (in which OM was administered on alternate days), in comparison with daily OM administration, resulted in stronger heart muscle tissues. Notably, each cycle of administration/withdrawal of the exemplary myotrope appears to trigger productive growth of heart muscle and dilute the negative side effects.

The results of the present study further indicate that the effects of the intermittent administration strategy are not limited to OM only but universal for myotropes in general.

Accordingly, in some aspects, the present disclosure is directed to a method of treating, ameliorating, and/or preventing a heart failure in a subject in need thereof.

In some aspects, the present disclosure is directed to a kit for treating, ameliorating and/or preventing a heart failure in a subject in need thereof.

In some aspects, the present disclosure is directed to a method of promoting the productive growth of a heart muscle.

In some embodiments, the present disclosure is directed to a kit for promoting the productive growth of a heart muscle.

The present disclosure is not limited to omecamtiv mecarbil itself, but applies to any myotropes or cardiac sarcomere activators described elsewhere herein and/or known in the art.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

As used herein, the term “productive growth” of a heart muscle refers to the long-term and sustained improvements in the contractile strength of the heart muscle, such as increased contractile strength lasting for about 1 week or more, about 2 weeks or more, about 3 weeks or more, about 1 month or more, about 2 months or more, about 3 months or more, about 6 months or more, or about 1 year or more.

As used herein, the term “contractile strength” of heart muscle means the force produced by a contraction of the heart muscle divided by the cross-sectional area (CSA) of the heart muscle.

As used herein, the terms “myotropes” or “cardiac myotropes” or “cardiac sarcomere activators” refers to therapeutic compounds that are able to treat or alleviate low cardiac output by acting on the sarcomere of the heart, such as myosin, actin, the associated regulatory proteins, or other structural elements of the sarcomere through calcium-independent mechanisms. The “myotropes” or “cardiac myotropes” as used herein include myosin activators, such as omecamtiv mecarbil. The terms “myotrope” and “cardiac myotrope” are also discussed in Psotka et al., J Am Coll Cardiol. 2019 May 14; 73 (18): 2345-2353, the entirety of which is hereby incorporated by reference. In certain embodiments, the myotrope sensitizes cardiac myofilaments to Ca²⁺. In certain embodiments, the myotrope activates troponin or tropomyosin. In certain embodiments, the myotrope directly activates the cardiac myosin. In certain embodiments, the myotrope increases the availability of force-producing cardiac myosins by perturbing thick filament structure or thick filament-associated proteins. Non-limiting examples of myotropes also include levosimendan, pimobendan, SR-33805, an HNO donor, CXL-1020, APD418 (beta3-AR antagonist), and/or danicamtiv.

In certain embodiments, the myotrope directly reacts with myosin and/or stabilizes the pre-powerstroke conformation of myosin facilitating the transition to the actin-bound state. In certain embodiments, the myotrope is an activator of cardiac myosin. In certain embodiments, the myotrope targets one or more of myosin, actin, troponin, tropomyosin, myosin regulatory light chain, myosin essential light chain, and myosin binding protein C. In certain embodiments, the myotrope activates one or more of myosin, actin, troponin, and tropomyosin. In certain embodiments, the myotrope is omecamtiv mecarbil (Methyl 4-[(2-fluoro-3-{[N-(6-methylpyridin-3-yl)carbamoyl]amino}phenyl)methyl]piperazine-1-carboxylate). Any form of omecamtiv mecarbil, including the free base, any pharmaceutically acceptable salt thereof (such as but not limited to a hydrochloride salt, a dihydrochloride salt, any solvate (such as but not limited to a hydrate) of any of the foregoing, and mixtures thereof in any ratio is contemplated herein. Omecamtiv mecarbil (also known as AMG 423, CK-1827452) is a cardiac myosin activator that increases cardiac contractility by selectively and directly activating the enzymatic domain of the cardiac myosin heavy chain, the force-generating motor protein of the cardiac sarcomere, without increasing cardiac myocyte intracellular calcium. This agent increases the left ventricular systolic ejection time (SET) without changing the velocity of contraction (dP/dt) or increasing the heart rate. Additionally, left ventricular filling pressures, left atrial pressures, and total peripheral vascular resistance decreased, providing evidence that prolongation of SET and increased systolic function can favorably impact the hemodynamics that drive HF symptoms. The salutary effects of OM were achieved without noticeable effect upon myocardial oxygen uptake, blood pressure, or coronary blood flow.

Abbreviations used herein: OM: omecamtiv mecarbil; EHT: engineered heart tissues; HFrEF: heart failure with reduced ejection fraction; LV: left ventricle; hiPSC-CM: human induced pluripotent stem cell-derived cardiomyocyte; DMEM: Dulbecco's modified eagle medium; DPBS: Dulbecco's phosphate-buffered saline; FBS: fetal bovine serum; NEAA: non-essential amino acids; DMSO: dimethyl sulfoxide; aHCF: adult human cardiac fibroblast; TTP: time to peak; RT50: time to 50% relaxation.

Method of Treating, Ameliorating and/or Preventing Heart Failure

In some embodiments, the instant disclosure is directed to a method of treating, ameliorating, and/or preventing a heart failure in a subject in need thereof.

In some embodiments, the method includes administering to the subject an effective amount of a myotrope using a dosing scheme having an administration-withdrawal cycle.

In some embodiments, the dosing scheme includes administering the myotrope at a frequency of less than two doses per day, such as once about every 24 hours (1 day), every 25 hours, every 26 hours, every 27 hours, every 28 hours, every 29 hours, every 30 hours, every 31 hours, every 32 hours, every 33 hours, every 34 hours, every 35 hours, every 36 hours, every 37 hours, every 38 hours, every 39 hours, every 40 hours, every 41 hours, every 42 hours, every 43 hours, every 44 hours, every 45 hours, every 46 hours, every 47 hours, every 48 hours (2 days), every 2.25 days, every 2.5 days, every 2.75 days, every 3 days, every 3.25 days, every 3.5 days, every 3.75 days, every 4 days, every 4.25 days, every 4.5 days, every 4.75 days, every 5 days, every 5.25 days, every 5.5 days, every 5.75 days, every 6 days, every 6.25 days, every 6.5 days, every 6.75 days, every 7 days, every 7.25 days, every 7.5 days, every 7.75 days, every 8 days, every 8.25 days, every 8.5 days, every 8.75 days, every 9 days, every 9.25 days, every 9.5 days, every 9.75 days, every 10 days, every 10.25 days, every 10.5 days, every 10.75 days, every 11 days, every 11.25 days, every 11.5 days, every 11.75 days, every 12 days, every 12.25 days, every 12.5 days, every 12.75 days, every 13 days, every 13.25 days, every 13.5 days, every 13.75 days, every 14 days, every 14.25 days, every 14.5 days, every 14.75 days, every 15 days, every 15.25 days, every 15.5 days, every 15.75 days, every 16 days, every 16.25 days, every 16.5 days, every 16.75 days, every 17 days, every 17.25 days, every 17.5 days, every 17.75 days, every 18 days, every 18.25 days, every 18.5 days, every 18.75 days, every 19 days, every 19.25 days, every 19.5 days, every 19.75 days, every 20 days, every 20.25 days, every 20.5 days, every 20.75 days, every 21 days, every 21.25 days, every 21.5 days, every 21.75 days, every 22 days, every 22.25 days, every 22.5 days, every 22.75 days, every 23 days, every 23.25 days, every 23.5 days, every 23.75 days, every 24 days, every 24.25 days, every 24.5 days, every 24.75 days, every 25 days, every 25.25 days, every 25.5 days, every 25.75 days, every 26 days, every 26.25 days, every 26.5 days, every 26.75 days, every 27 days, every 27.25 days, every 27.5 days, every 27.75 days, every 28 days, every 28.25 days, every 28.5 days, every 28.75 days, every 29 days, every 29.25 days, every 29.5 days, every 29.75 days, every 30 days (1 month), or any combination, fraction, or multiple thereof.

In some embodiments, the dosing scheme includes administering to the subject a placebo in the day(s) or week(s) when the subject is not supposed to be administered with the myotrope. Due to that the dosing intervals according to some non-limiting embodiments herein are longer than those used for conventional administrations of myotropes, the subject may neglect to take one or more doses of myotropes, resulting in reduced effect of the dosing scheme. The addition of placebo doses allows the subject to take medication on a more frequent schedule, such as daily or weekly, and therefore the reduced chance of neglecting to take medicine. In some embodiments, doses of the myotrope and the placebo are arranged, such as in a pill pack or a blister pack, to reduce the possibility of confusion.

In some embodiments, each dose of the myotrope include administering a myotrope dose. In some embodiments, the dose is of about 100 mg or lower, about 75 mg or lower, about 50 mg or lower, about 37.5 mg or lower, about 25 mg or lower, about 12.5 mg or lower, about 6.25 mg or lower. In some embodiments, the dose is of about 75 mg or higher, about 50 mg or higher, about 37.5 mg or higher, about 25 mg or higher, about 12.5 mg or higher, about 6.25 mg or higher. In some embodiments, the myotrope dose is an oral myotrope dose.

In some embodiments, the myotrope is administered according to the administration-withdrawal cycle such that the plasma concentration of the myotrope in the subject cycles between above a threshold concentration and below the threshold concentration during cycles of the administration. In some embodiments, the threshold concentration is about 1000 ng/ml or less, for example about 900 ng/ml, about 800 ng/ml, about 700 ng/ml, about 600 ng/ml, about 500 ng/ml, about 400 ng/ml, about 300 ng/ml, about 200 ng/mL, about 150 ng/mL, about 100 ng/ml, about 90 ng/ml, about 80 ng/ml, about 70 ng/mL, about 60 ng/ml, or about 50 ng/mL. In some embodiments, the threshold concentration is lower than about 1000 ng/mL, about 900 ng/ml, about 800 ng/mL, about 700 ng/mL, about 600 ng/mL, about 500 ng/ml, about 400 ng/ml, about 300 ng/ml, about 200 ng/mL, about 150 ng/mL, about 100 ng/ml, about 90 ng/ml, about 80 ng/ml, about 70 ng/ml, about 60 ng/mL, or about 50 ng/mL. In some embodiments, the threshold concentration is higher than about 900 ng/mL, about 800 ng/mL, about 700 ng/ml, about 600 ng/mL, about 500 ng/ml, about 400 ng/mL, about 300 ng/ml, about 200 ng/ml, about 150 ng/mL, about 100 ng/mL, about 90 ng/ml, about 80 ng/ml, about 70 ng/mL, about 60 ng/ml, or about 50 ng/ml.

In some embodiments, the myotrope is administered according to the administration-withdrawal cycle such that the plasma concentration of the myotrope in the subject cycles above a first threshold concentration and below a second threshold concentration during cycles of administration. In some embodiments, the first threshold concentration is an effective activating threshold concentration above which the myotrope is able to acutely enhance cardiac contraction. In some embodiments, the second threshold concentration is an effective deactivation threshold concentration below which the myotrope does not acutely affect cardiac contraction.

In some embodiments, the first threshold concentration is about 1000 ng/ml or less, such as about 900 ng/mL, about 800 ng/mL, about 700 ng/mL, about 600 ng/ml, about 500 ng/mL, about 400 ng/mL, about 300 ng/ml, about 200 ng/ml, about 150 ng/ml, about 100 ng/mL, about 90 ng/ml, about 80 ng/mL, about 70 ng/ml, about 60 ng/ml, or about 50 ng/mL. In some embodiments, the first threshold concentration is lower than about 1000 ng/mL, about 900 ng/mL, about 800 ng/mL, about 700 ng/mL, about 600 ng/ml, about 500 ng/mL, about 400 ng/mL, about 300 ng/mL, about 200 ng/ml, about 150 ng/ml, about 100 ng/mL, about 90 ng/mL, about 80 ng/mL, about 70 ng/mL, about 60 ng/ml, or about 50 ng/mL. In some embodiments, the first threshold concentration is higher than about 900 ng/mL, about 800 ng/mL, about 700 ng/mL, about 600 ng/ml, about 500 ng/mL, about 400 ng/mL, about 300 ng/mL, about 200 ng/ml, about 150 ng/ml, about 100 ng/ml, about 90 ng/mL, about 80 ng/mL, about 70 ng/mL, about 60 ng/mL, or about 50 ng/ml.

In some embodiments, the effective deactivating threshold concentration is about 500 ng/ml or less, such as about 400 ng/ml, about 300 ng/ml, about 200 ng/ml, about 150 ng/ml, about 100 ng/ml, about 50 ng/ml, or about 25 ng/ml or lower. In some embodiments, the effective deactivating threshold concentration is lower than about 500 ng/ml, about 400 ng/ml, about 300 ng/ml, about 200 ng/ml, about 150 ng/ml, about 100 ng/ml, about 50 ng/ml, or about 25 ng/ml. In some embodiments, the effective deactivating threshold concentration is higher than about 400 ng/ml, about 300 ng/ml, about 200 ng/ml, about 150 ng/ml, about 100 ng/ml, about 50 ng/ml, or about 25 ng/ml. In some embodiments, the first threshold is higher than the second threshold.

In some embodiments, the dosing scheme of the myotrope further includes, before the administration-withdrawal cycle, an acceleration phase. In some embodiments, the first one or several administration phases of the administration-withdrawal cycle is replaced with the acceleration phase(s). In one acceleration phase, the subject is administered with the myotrope at a higher doses and/or with a higher administration frequency than those used in the administration phase of the administration-withdrawal cycle such that the plasma concentration of the myotrope is able to reach the threshold concentration, the first threshold concentration, and/or the effective activating threshold concentration of the previous paragraphs before the first withdrawal phase starts. In some embodiments, the administration of myotrope at a higher doses and/or with a higher administration frequency associated with the acceleration phase is continued in subsequent administration phases.

In some embodiments, the myotrope includes omecamtiv mecarbil (OM), APD418 (beta3-AR antagonist), danicamtiv, levosimendan, or combinations thereof. In some embodiments, the myotrope includes OM. In some embodiments, the only myotrope administered to the subject is OM.

In some embodiments, the method causes productive growth of a heart muscle in the subject.

In some embodiments, the method does not reduce the diastolic performance of the heart of the subject.

In some embodiments, the subject is a mammal. In some embodiments, the subject is a human.

Kit for Treating, Ameliorating and/or Preventing Heart Failure

In some aspects, the present disclosure is directed to a kit for treating, ameliorating, and/or preventing a heart failure in a subject in need thereof. In some embodiments, the kit includes a myotrope; and a manual instructing that the myotrope is to be administered to a subject at an effective amount using a dosing scheme having an administration-withdrawal cycle.

In some embodiments, the kit further includes a placebo. In some embodiments, the kit further includes a pill pack or a blister pack. In some embodiments, the myotrope and placebo are arranged in the pill pack or the blister pack according to a predetermined order based on the dosing scheme.

In some embodiments, the manual provides an instruction to perform a method of treating, ameliorating, and/or preventing heart failure in a subject. In some embodiments, the method of treating, ameliorating, and/or preventing heart failure in a subject is the same as or similar to those described elsewhere herein.

### Method of Promoting Productive Growth of Heart Muscle

In some aspects, the present disclosure is directed to a method of promoting productive growth of a heart muscle. In some embodiments, the method includes repeatedly contacting the heart muscle with a myotrope under an intermittent cycle.

In some embodiments, the heart muscle is contacted with the myotrope at a frequency of less than two doses per day, such as once about every 24 hours (1 day), every 25 hours, every 26 hours, every 27 hours, every 28 hours, every 29 hours, every 30 hours, every 31 hours, every 32 hours, every 33 hours, every 34 hours, every 35 hours, every 36 hours, every 37 hours, every 38 hours, every 39 hours, every 40 hours, every 41 hours, every 42 hours, every 43 hours, every 44 hours, every 45 hours, every 46 hours, every 47 hours, every 48 hours (2 days), every 2.25 days, every 2.5 days, every 2.75 days, every 3 days, every 3.25 days, every 3.5 days, every 3.75 days, every 4 days, every 4.25 days, every 4.5 days, every 4.75 days, every 5 days, every 5.25 days, every 5.5 days, every 5.75 days, every 6 days, every 6.25 days, every 6.5 days, every 6.75 days, every 7 days, every 7.25 days, every 7.5 days, every 7.75 days, every 8 days, every 8.25 days, every 8.5 days, every 8.75 days, every 9 days, every 9.25 days, every 9.5 days, every 9.75 days, every 10 days, every 10.25 days, every 10.5 days, every 10.75 days, every 11 days, every 11.25 days, every 11.5 days, every 11.75 days, every 12 days, every 12.25 days, every 12.5 days, every 12.75 days, every 13 days, every 13.25 days, every 13.5 days, every 13.75 days, every 14 days, every 14.25 days, every 14.5 days, every 14.75 days, every 15 days, every 15.25 days, every 15.5 days, every 15.75 days, every 16 days, every 16.25 days, every 16.5 days, every 16.75 days, every 17 days, every 17.25 days, every 17.5 days, every 17.75 days, every 18 days, every 18.25 days, every 18.5 days, every 18.75 days, every 19 days, every 19.25 days, every 19.5 days, every 19.75 days, every 20 days, every 20.25 days, every 20.5 days, every 20.75 days, every 21 days, every 21.25 days, every 21.5 days, every 21.75 days, every 22 days, every 22.25 days, every 22.5 days, every 22.75 days, every 23 days, every 23.25 days, every 23.5 days, every 23.75 days, every 24 days, every 24.25 days, every 24.5 days, every 24.75 days, every 25 days, every 25.25 days, every 25.5 days, every 25.75 days, every 26 days, every 26.25 days, every 26.5 days, every 26.75 days, every 27 days, every 27.25 days, every 27.5 days, every 27.75 days, every 28 days, every 28.25 days, every 28.5 days, every 28.75 days, every 29 days, every 29.25 days, every 29.5 days, every 29.75 days, every 30 days (1 month), or any combination, fraction, or multiple thereof.

In some embodiments, the concentration of the myotrope being contacted with the heart muscle cycles between above a threshold concentration and below the threshold concentration during the contact-withdrawal cycle. In some embodiments, the threshold concentration is about 1000 ng/mL or less, for example about 900 ng/mL, about 800 ng/ml, about 700 ng/ml, about 600 ng/mL, about 500 ng/ml, about 400 ng/mL, about 300 ng/ml, about 200 ng/mL, about 150 ng/mL, about 100 ng/mL, about 90 ng/ml, about 80 ng/ml, about 70 ng/mL, about 60 ng/mL, or about 50 ng/ml. In some embodiments, the threshold concentration is lower than about 1000 ng/ml, about 900 ng/mL, about 800 ng/mL, about 700 ng/ml, about 600 ng/ml, about 500 ng/ml, about 400 ng/ml, about 300 ng/mL, about 200 ng/ml, about 150 ng/ml, about 100 ng/mL, about 90 ng/ml, about 80 ng/ml, about 70 ng/mL, about 60 ng/ml, or about 50 ng/mL. In some embodiments, the threshold concentration is higher than about 900 ng/mL, about 800 ng/mL, about 700 ng/ml, about 600 ng/mL, about 500 ng/ml, about 400 ng/mL, about 300 ng/mL, about 200 ng/ml, about 150 ng/mL, about 100 ng/ml, about 90 ng/ml, about 80 ng/mL, about 70 ng/ml, about 60 ng/ml, or about 50 ng/mL.

In some embodiments, the concentration of the myotrope being contacted with the heart muscle cycles above a first threshold concentration and below a second threshold concentration during the contact-withdrawal cycle. In some embodiments, the first threshold concentration is an effective activating threshold concentration above which the myotrope is able to enhance contraction of the heart muscle. In some embodiments, the second threshold concentration is an effective deactivation threshold concentration below which the myotrope does not acutely affect cardiac contraction.

In some embodiments, the first threshold concentration is about 1000 ng/ml or less, such as about 900 ng/mL, about 800 ng/mL, about 700 ng/mL, about 600 ng/ml, about 500 ng/mL, about 400 ng/mL, about 300 ng/mL, about 200 ng/mL, about 150 ng/ml, about 100 ng/ml, about 90 ng/ml, about 80 ng/mL, about 70 ng/mL, about 60 ng/ml, or about 50 ng/mL. In some embodiments, the first threshold concentration is lower than about 1000 ng/ml, about 900 ng/mL, about 800 ng/mL, about 700 ng/ml, about 600 ng/ml, about 500 ng/mL, about 400 ng/mL, about 300 ng/mL, about 200 ng/ml, about 150 ng/mL, about 100 ng/mL, about 90 ng/ml, about 80 ng/mL, about 70 ng/mL, about 60 ng/ml, or about 50 ng/mL. In some embodiments, the first threshold concentration is higher than about 900 ng/mL, about 800 ng/ml, about 700 ng/ml, about 600 ng/ml, about 500 ng/ml, about 400 ng/mL, about 300 ng/mL, about 200 ng/ml, about 150 ng/ml, about 100 ng/ml, about 90 ng/mL, about 80 ng/ml, about 70 ng/ml, about 60 ng/ml, or about 50 ng/mL.

In some embodiments, the effective deactivating threshold concentration is about 500 ng/ml or less, such as about 400 ng/ml, about 300 ng/ml, about 200 ng/ml, about 150 ng/ml, about 100 ng/ml, about 50 ng/ml, or about 25 ng/ml or lower. In some embodiments, the effective deactivating threshold concentration is lower than about 500 ng/ml, about 400 ng/ml, about 300 ng/ml, about 200 ng/ml, about 150 ng/ml, about 100 ng/ml, about 50 ng/ml, or about 25 ng/ml. In some embodiments, the effective deactivating threshold concentration is higher than about 400 ng/ml, about 300 ng/ml, about 200 ng/ml, about 150 ng/ml, about 100 ng/ml, about 50 ng/ml, or about 25 ng/ml. In some embodiments, the first threshold is higher than the second threshold. In some embodiments, the first threshold is higher than the second threshold.

In some embodiments, the dosing scheme of the myotrope further includes, before the administration-withdrawal cycle, an acceleration phase. In some embodiments, the first administration phase of the administration-withdrawal cycle is replaced with the acceleration phase. In the acceleration phase, the subject is administered with the myotrope at a higher does and/or with a higher administration frequency than those used in the administration phase of the administration-withdrawal cycle such that the plasma concentration of the myotrope is able to reach the threshold concentration, the first threshold concentration, and/or the effective activating threshold concentration of the previous paragraphs before the first withdrawal phase starts.

In some embodiments, the myotrope includes omecamtiv mecarbil (OM), APD418 (beta3-AR antagonist), danicamtiv, levosimendan, or combinations thereof. In some embodiments, the myotrope includes OM. In some embodiments, the only myotrope contacted with the heart muscle is OM.

In some embodiments, the heart muscle is in an engineered heart tissue. In some embodiments, the heart muscle is in the heart of a subject. In some embodiments, the subject is a mammal. In some embodiments, the subject is a human.

### Kit for Promoting Productive Growth of Heart Muscle

In some aspects, the present disclosure is directed to a kit for causing growth of a heart muscle. In some embodiments, the kit includes a myotrope; and a manual instructing that the myotrope is to be intermittently contacted with the heart muscle.

In some embodiments, the manual provides an instruction to perform a method of promoting productive growth of heart muscle. In some embodiments, the method of treating, ameliorating and/or preventing heart failure in a subject is the same as or similar to those described elsewhere herein.

### Combination Therapies

The compounds useful within the methods described herein can be used in combination with one or more additional therapeutic agents useful for treating, ameliorating and/or preventing heart failure. These additional therapeutic agents may comprise compounds that are commercially available or synthetically accessible to those skilled in the art. These additional therapeutic agents are known to treat or reduce the symptoms, of a heart failure.

In various embodiments, a synergistic effect is observed when a compound as described herein is administered with one or more additional therapeutic agents or compounds. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

During the “administration” portion of the administration-withdrawal cycle, the administration of the myotrope sometimes needs to be able to achieve the goal of improving the contractility of the heart of the subject before the “withdrawal” portion of the cycle takes place. Choice of administration, dosage and/or formulation sometimes helps achieving this goal.

The regimen of administration may affect what constitutes an effective amount. The therapeutic formulations may be administered to the subject either prior to or after the onset of a heart failure. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a heart failure in the patient. An effective amount of the therapeutic compound necessary to achieve a therapeutic effect may vary according to factors such as the state of the disease or disorder in the patient; the age, sex, and weight of the patient; and the ability of the therapeutic compound to treat a heart failure in the patient. Dosage regimens may be adjusted to provide the optimum therapeutic response. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required based on the knowledge provided by the instant specification. For example, the physician or veterinarian could start doses of the compounds described herein employed in the pharmaceutical composition at levels lower than that required in order to achieve the desired therapeutic effect and gradually increase the dosage until the desired effect is achieved, before starting the administration-withdrawal cycle.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more during the “administration” portion of the administration-withdrawal cycle. In other embodiments, the compositions described herein are administered to the patient in range of dosages that include, but are not limited to, every 36 hours, every two days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account, and based on the knowledge provided by the instant specification.

The compound(s) described herein for administration during the “administration” portion of the administration-withdrawal cycle may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein during the “administration” portion of the administration-withdrawal cycle is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% □-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

### Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patents Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

During the “administration” portion of the administration-withdrawal cycle, the administration of the myotrope sometimes needs to be able to achieve the goal of improving the contractility of the heart of the subject before the “withdrawal” portion of the cycle takes place. Use of controlled release formulations as well as choice of drug delivery system sometimes helps achieving this goal.

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a heart failure in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

Omecamtiv mecarbil (OM) was discovered in the early 2000s as one of the first molecules in its class to specifically target cardiac myosin for treating heart failure (HF). OM stimulates cardiac myosin ATPase activity by elevating the rate of actin-myosin cross-bridge formation and this results in increased contractility. Moreover, OM increases the time myosin is strongly bound to actin while keeping a subset of myosin heads in a weak actin affinity state. As a result, OM slows force development, prolongs heart systolic ejection time, and increases diastolic baseline muscle tone and calcium sensitivity of force production in a dose-dependent manner. OM as a cardiac myosin activator differs from classical positive inotropes because it does not affect myocyte calcium homeostasis or myocardial oxygen consumption. However, other evidence suggests that OM induces mitochondrial oxidation and mismatch between ATP and reactive oxygen species production while mitochondrial redox capacity remains unchanged. There is also debate on the role played by OM in increased myocardial oxygen consumption and impaired cardiac efficiency in pigs.

The promising in vitro and experimental properties of OM led to large-scale clinical trials for patients with heart failure with reduced ejection fraction (HFrEF), chronic heart failure, and acute heart failure. Although OM seemed to be well tolerated across trials, the drug yielded only limited primary endpoint improvements (time to first HF event, health-related quality of life, cardiac function, and dyspnea). The limited primary outcome benefits (incidence rate of heart failure or cardiovascular death) were especially disappointing for the Global Approach to Lowering Adverse Cardiac Outcomes Through Improving Contractility in Heart Failure (GALATIC-HF) phase 3 randomized clinical trial given the size of the study. It was only when a post hoc analysis focusing on the subset of most severe HF patients was performed that a clinically meaningful improvement in the time to first HF or cardiovascular death was found.

The surprisingly lackluster clinical trial performances of OM despite its potent effect on cardiomyocyte contractility led to the study described herein (“the present study”), which searched for alternative OM dosing strategies that might maximize its systolic benefits at minimal cost in diastolic performance, relaxation kinetics, and other important functional parameters of the myocardium.

It was hypothesized that optimal long-term effects of OM could be obtained if given in an intermittent or ‘pulsed’ manner instead of a constant dose. The current OM clinical trials rely on a pharmacokinetic-guided dose titration strategy, essentially providing a near constant plasma OM concentration via two daily doses (Teerlink et al., JACC. Heart Fail. 8, 329-340 (2020)).

The present study tested this radically different dosing strategy in vitro by administering OM on alternating days to engineered heart tissues (EHTs) formed from induced pluripotent stem cell-derived cardiomyocytes. Pulsed OM administration was compared to a constant concentration regime more closely resembling clinical trials. The schemes were also compared while being applied to an in vitro model of genetic heart failure.

### Example 2: Materials and Methods

### Materials

Dulbecco's modified eagle medium (DMEM), Dulbecco's phosphate-buffered saline (DPBS), RPMI 1640 medium, fetal bovine serum (FBS), penicillin-streptomycin (P/S, 10,000 U/mL), B-27 supplements with and without insulin, non-essential amino acids (NEAA), L-glutamine, sodium pyruvate, and TrypLE were purchased from Thermofisher Scientific. Dimethyl sulfoxide (DMSO) came from Sigma Aldrich. Omecamtiv mecarbil was from Cayman Chemical. mTESR media, CHIR99021, and IWP4 were from STEMCELL Technologies.

### Tissue Procurement, Processing, and Cryostat Sectioning

Fresh porcine hearts were purchased from J Latella & Sons (West Haven, CT). After the heart were purchased, they were immediately submerged in ice-cold DPBS with 5% Penicillin-Streptomycin during transport. For heart processing, a surgical scalpel is used to trim away the atria and right ventricle, leaving only the left ventricle (LV). LV was cut from the base end to the apex so it could be unrolled into a roughly rectangular block. Then endocardium and epicardium portion of LV were removed. In the end, only the LV free wall was kept before it was further trimmed, cut into 3 cm square blocks, flash frozen on crushed dry ice, and stored in the deep freezer.

For cryostat sectioning of LV blocks into 150 μm-thick longitudinal slices, a cryostat microtome (Leica CM3050 S) was used. The block was mounted onto the sample disk with Tissue-Tek compound (Sakura) at −13° C. object temperature. A low-profile diamond microtome blade was used to cut the block at a 7.5-degree angle. Once cut, the slices were labelled and stored in a large petri dish in the deep freezer.

Maintenance and Differentiation of hiPSC-CMS

A healthy control stem cell line (GM23338, Coriell Institute) and an inherited dilated cardiomyopathy line with α-tropomyosin mutation were cultured in mTESR until close to 100% confluency. A commonly used differentiated protocol was adopted and used (Lian, et al. Nat. Protoc. 8, 162-175 (2013)). At day 0, 15 μM CHIR99021 was added for 24 h, and 5 μM IWP4 was added in media on day 3 for 48 h. Cells were cultured in RPMI with B-27 minus insulin supplement until day 9. Afterwards, RPMI with B-27 plus insulin was used. A 4-day cardiomyocyte purification process was initiated at day 12 with 4 mM lactate in RPMI without glucose. Cells were used for EHT seeding on day 18.

### Fabrication, Seeding, and Maintenance of Engineered Heart Tissues (EHTs)

To produce EHTs, frozen LV slices were laid flat on top of cover glass slides (25 mm diameter, Bellco Glass) held in a 3D-printed cover glass holder. Slices are then laser cut into 2.5 mm×6 mm rectangular strips and submerged in DPBS. The strips are incubated in lysis buffer with 10 mM Tris and 0.1% 0.5 M EDTA for two hours. EHT holders are prepared from 0.1-inch-thick Teflon sheets (ePlastics). After laser cutting, EHT Teflon holders are cleaned and autoclaved before scaffolds are fixated onto the EHT holders. Assembled scaffolds are decellularized in 0.5% wt/vol sodium dodecyl sulfate in DPBS for 45 min at 50 RPM on a plate shaker. Finally, the scaffolds are incubated overnight in an incubation media (DMEM, 10% FBS, and 2% P/S) before seeding.

For EHT seeding, hiPS-CMs were dissociated with TrypLE and adult human cardiac fibroblasts (aHCFs, 306-05A, PromoCell) were thawed from liquid nitrogen storage. A total of one million cells was seeded onto each tissue, with 90% hiPSC-CMs and 10% aHCFs. Seeding media was made ahead of time with DMEM, 10% FBS, 1% P/S, 1% NEAA, 1% L-glutamine, and 1% sodium pyruvate. After seeding, the tissues were maintained in DMEM supplemented with B-27 plus insulin. Media was changed every other day. The tissues were monitored for two weeks to allow proper cell attachment and contractile synchronization before experiments.

### Mechanical Testing and Data Analysis

A custom system was set up to characterize the biomechanical properties of EHTs. The system was comprised of a pair of linear actuators and a sensitive force transducer that could measure the mechanics of the tissues in real time. A custom 3D-printed bath surrounded with heating elements, thermistor probe, and a pair of platinum wires enabled the tissues to be studied in physiological temperature, under controlled electrical stimulation frequency, and continuous perfusion of Tyrode's solution. Tyrode's solution was freshly prepared for every experiment (140 mM NaCl, 5.4 mM KCl, 1.8 mM CaCl₂), 1 mM MgCl₂, 25 mM HEPES, and 10 mM glucose; pH 7.3). A custom MATLAB system was used for system control, data recording, and analysis. GraphPad Prism 9 was used for statistical analysis and data visualization.

### Example 3: Long-Term Constant Omecamtiv Mecarbil Dosing Provides Systolic Improvement at Significant Diastolic Costs

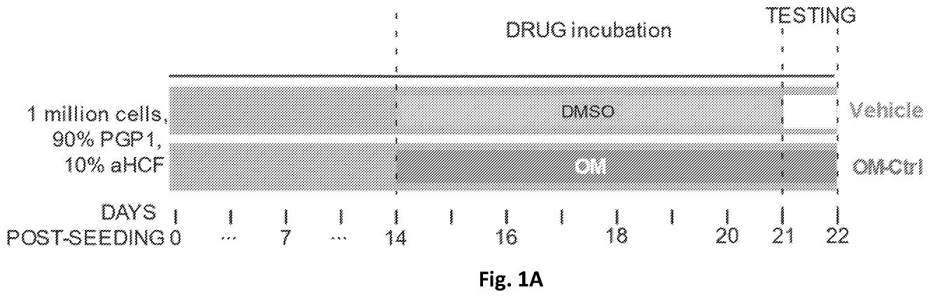

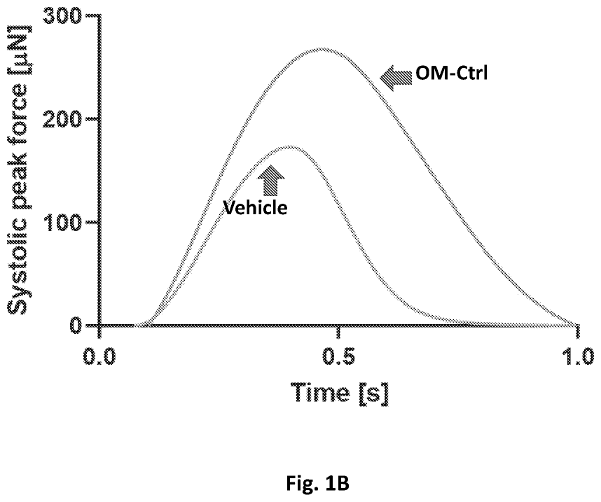

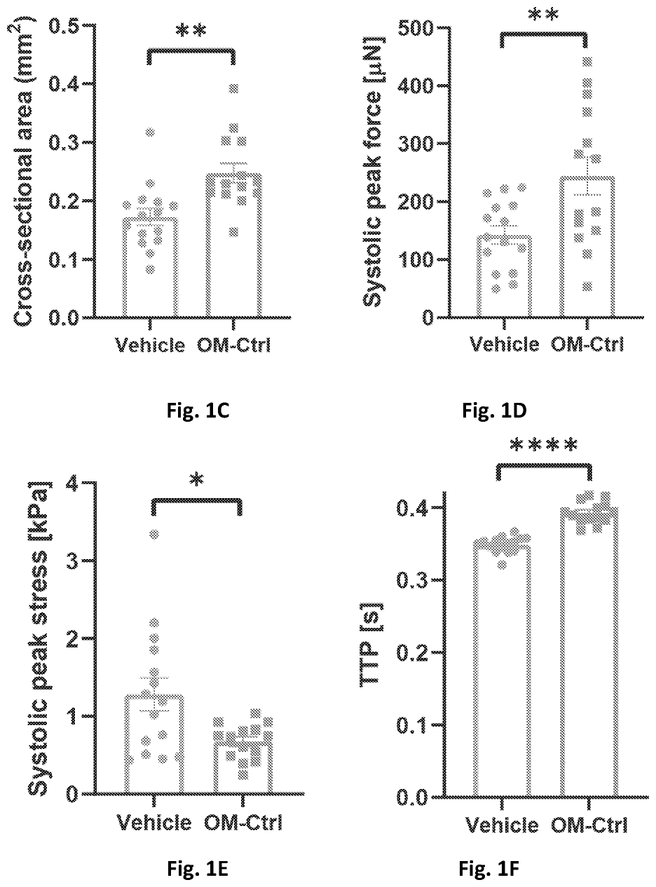

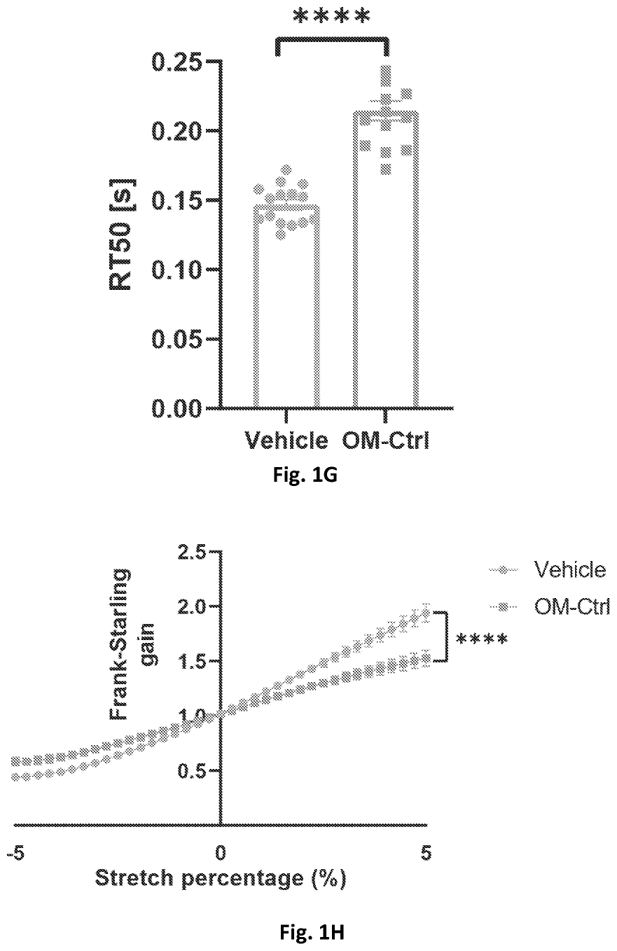

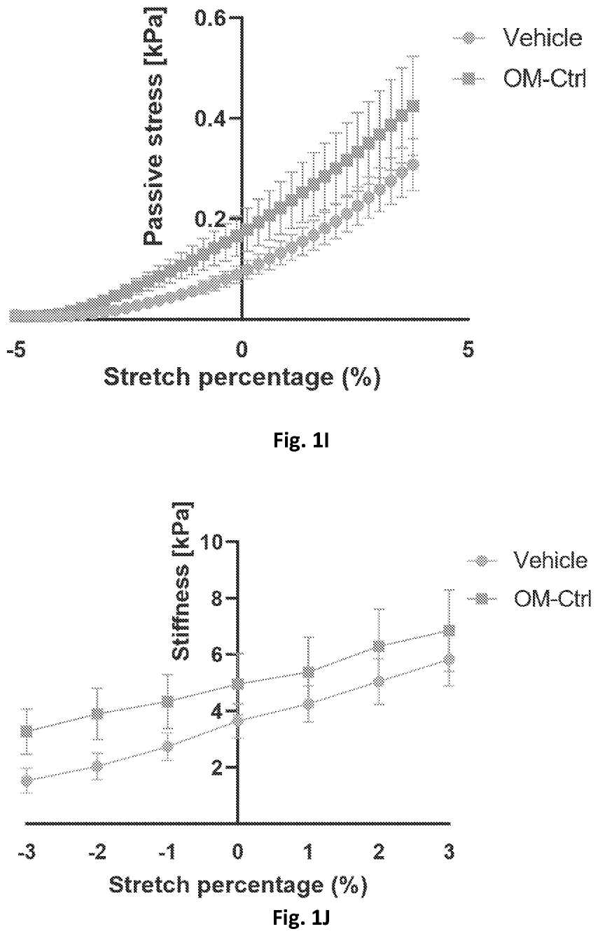

An initial experiment was performed to establish baseline chronic performance of OM in a manner resembling current clinical practice, that is, constant-concentration dosing administered over several days to human EHTs in vitro. EHTs were produced by seeding human induced pluripotent stem cell-derived cardiomyocytes (hiPSC-CMs) and adult cardiac fibroblasts at 9:1 ratio onto decellularized porcine myocardial scaffolds (Schwan et al. Sci. Rep. 6, 32068 (2016)). These hiPSC-CMs were derived from a healthy control line (GM23338, Coriell Institute). Tissues were incubated for two weeks to ensure proper cell attachment and contractile synchronization. Subsequently, EHTs were randomly assigned to two treatment groups. The first received a constant 0.5 μM OM dose (concentration to achieve half-maximal acute contractile increase per Shen et al. (J. Am. Heart Assoc. 10, (2021)) over 8 days (OM-Ctrl). The second group was a vehicle control, receiving DMSO over 8 days at a concentration equal to that of the OM-Ctrl group (vehicle). (FIGS. 1A-1J). After 8 days under treatment conditions, contractile performance of the OM-Ctrl tissues was measured in Tyrode's solution with the same 0.5 μM OM dose. In this way, behavior of OM-Ctrl tissues reflected both the chronic and acute effects of OM and approximates the scenario experienced by patients on a titrated OM dose.

Isometric twitch behavior in 1 Hz-stimulated EHTs was visibly different between OM-Ctrl and vehicle groups (FIG. 1B). OM-Ctrl treated tissues were stronger and slower. Moreover, cross-sectional area (CSA) measured by optical coherence tomography showed significant increase for OM-Ctrl, suggesting potential hypertrophy after long-term OM incubation (FIG. 1C). Although OM-Ctrl had significantly higher systolic peak force (FIG. 1D), the peak stress (force divided by CSA) was significantly lower (FIG. 1E). This reversal points out the possible reduction in effective systolic function per unit area even when the raw contractility had increased. What is more, OM-Ctrl tissues had large, highly significant increases in both time to peak (TTP, FIG. 1F) and time to 50% relaxation (RT50, FIG. 1G). OM also significantly worsened the ability of tissues to augment contractile force in response to stretch (FIG. 1H). Lastly, OM-Ctrl group had observable increases in both passive stress (FIG. 1I) and passive stiffness (FIG. 1J). Collectively, this controlled experiment demonstrates that under chronic treatment OM produces increased systolic force in EHTs but does so at the cost of slower kinetics, worsened length-dependent activation, and elevated diastolic stiffness. All observations were consistent with in vitro and pre-clinical reports on the effects of OM, suggesting a meaningful resemblance of the in vitro EHT model to the effects of OM in vivo.

### Example 4: Pulsed OM Dosing Provides Systolic Improvement Over Constant Dosing

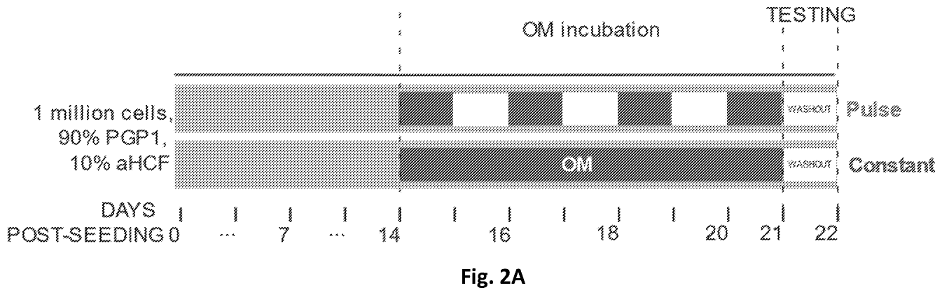

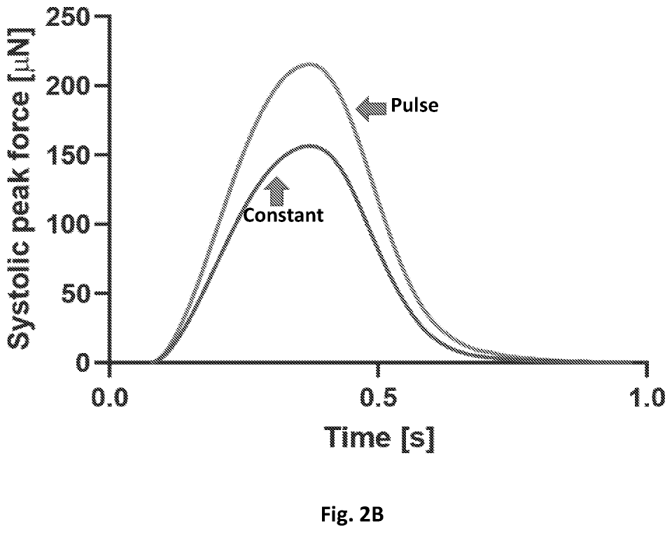

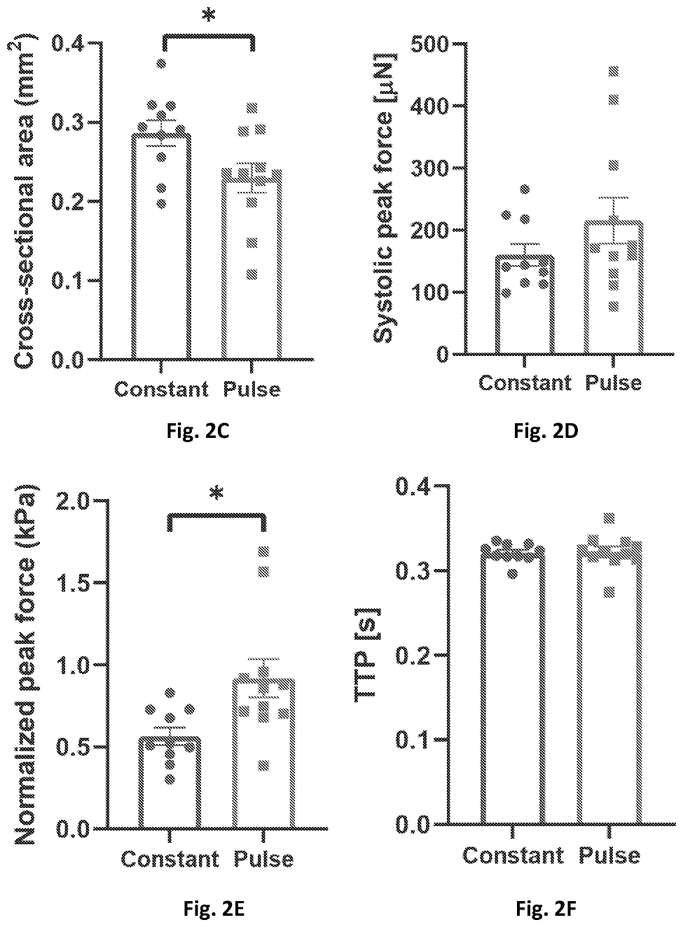

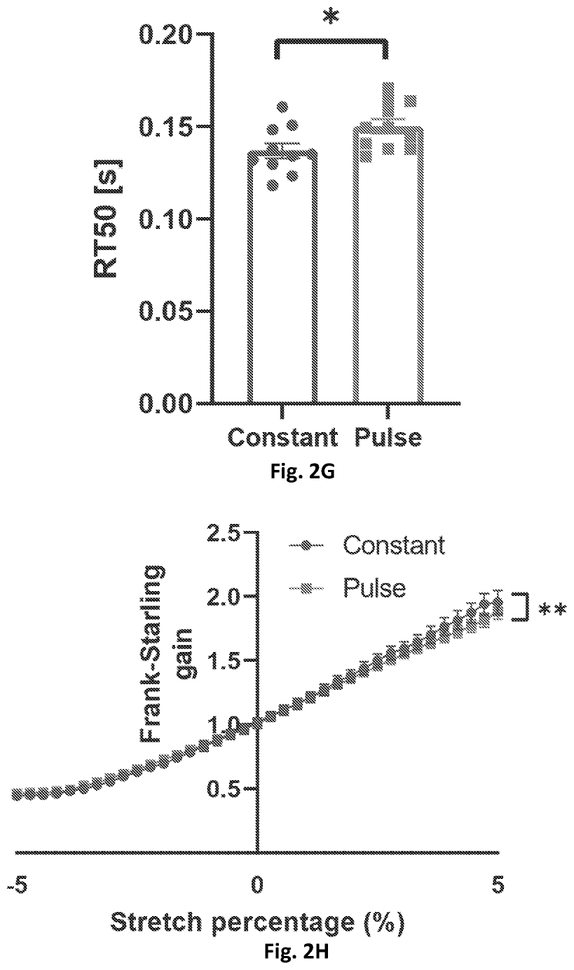

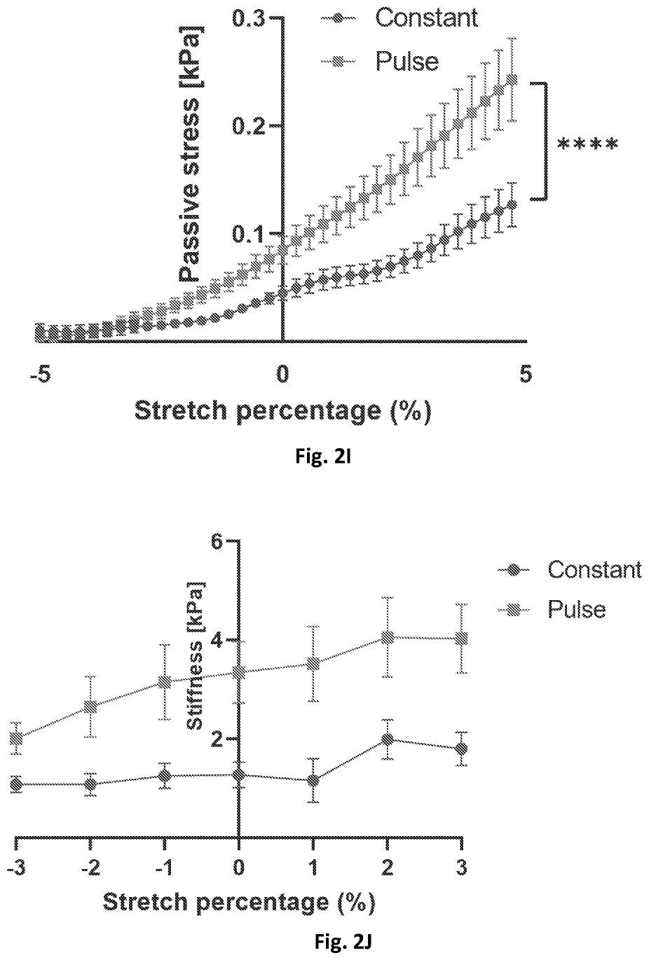

Having established the effects of a constant OM dosing regimen in the EHT system, the present study performed the intermittent dosing experiment to test the potential benefits of a pulsed administration strategy. In this experiment, one group of EHTs was randomly selected for incubation in 0.5 μM OM over 7 days while a second group was incubated in 0.5 μM OM and then drug-free media in alternating 24-hr periods, also for 7 days (constant and pulsed groups respectively, FIG. 2A). Both groups received a final 24-hr washout period in drug-free media so that only the sustained effects of OM would be measured. Under 1 Hz electrical pacing, sample twitches showed similar kinetics but the pulse group EHTs had higher systolic force (FIG. 2B). The pulse group had significantly lowered cross-sectional area (FIG. 2C), which translated a 34% higher systolic peak force into a significant 63% increase in systolic peak stress for the pulse group compared with the constant group (FIG. 2D-2E). Interestingly, both constant and pulse groups exhibited kinetic properties that were similar to the vehicle-treated EHTs in the previous experiment, with near identical TTP (FIG. 2F) and marginally increased RT50 in the pulse group (FIG. 2G). The two groups also showed similar augmented contraction in response to stretch, with shared behavior deviating only at higher levels of stretch (FIG. 2H). In terms of diastolic profiles, the pulse group showed higher passive stress and stiffness (FIG. 2I-2J).

### Example 5: Pulsed OM Dosing Provides Systolic Improvement Over Constant Dosing in a Mutant Dilated Cardiomyopathy Line E54K

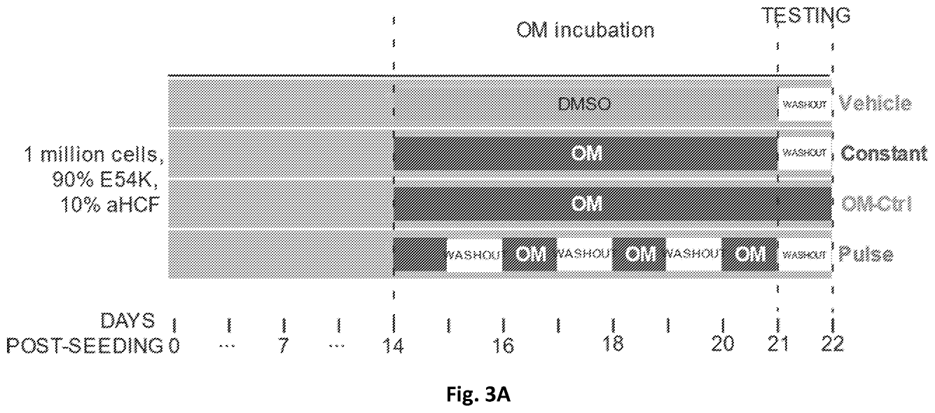

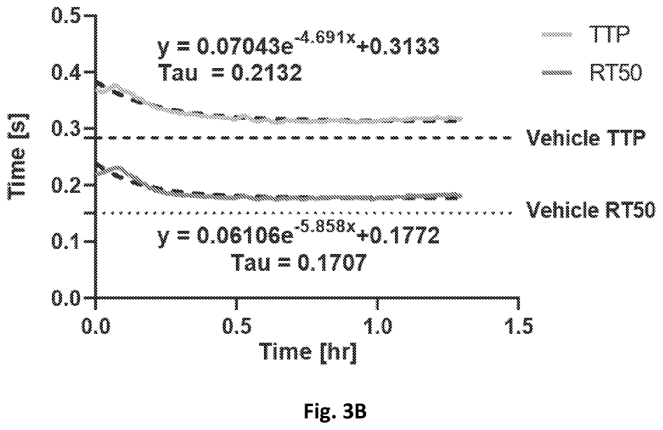

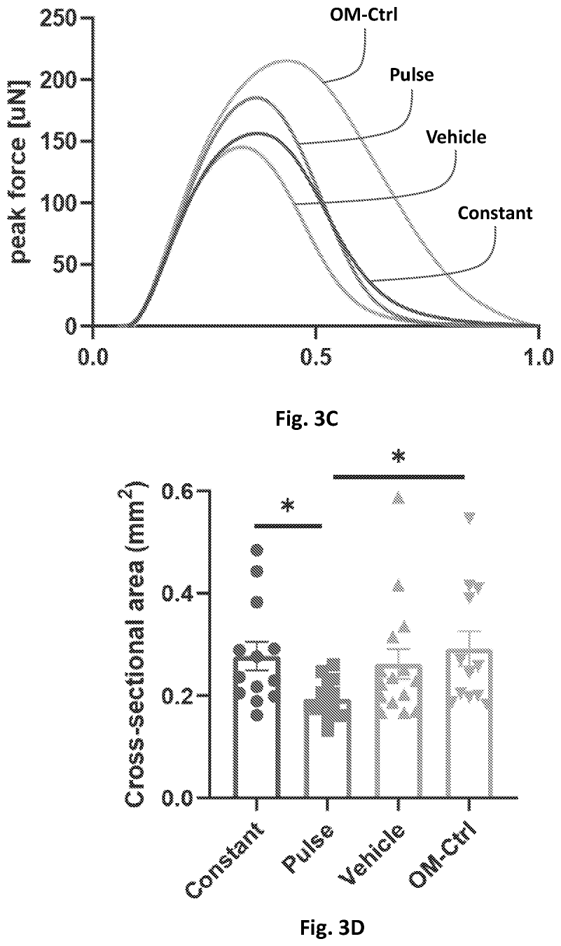

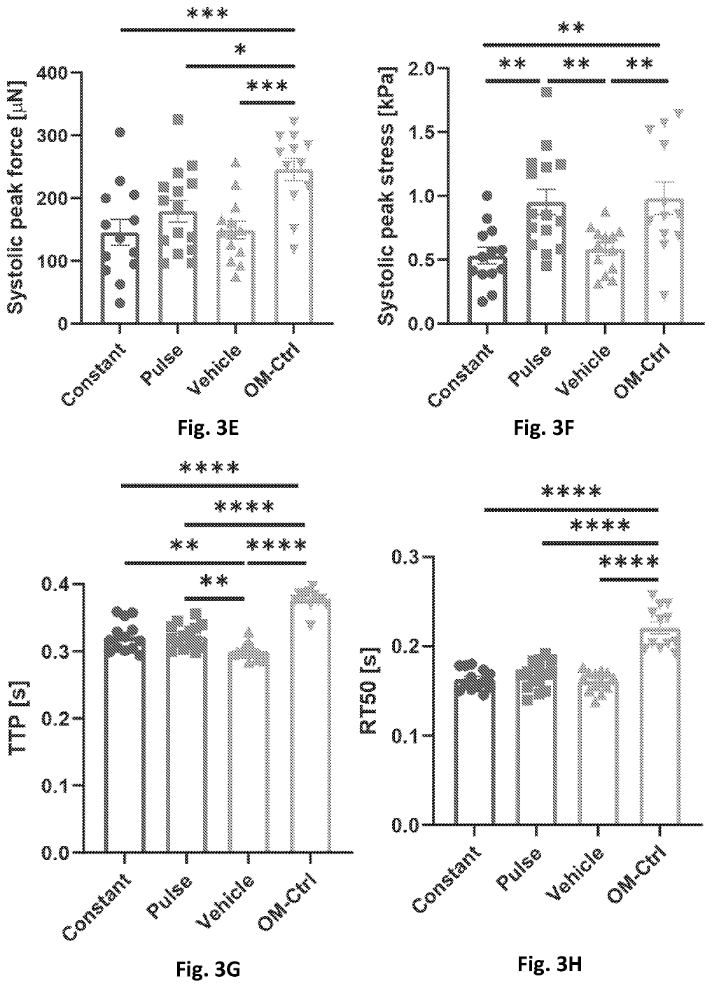

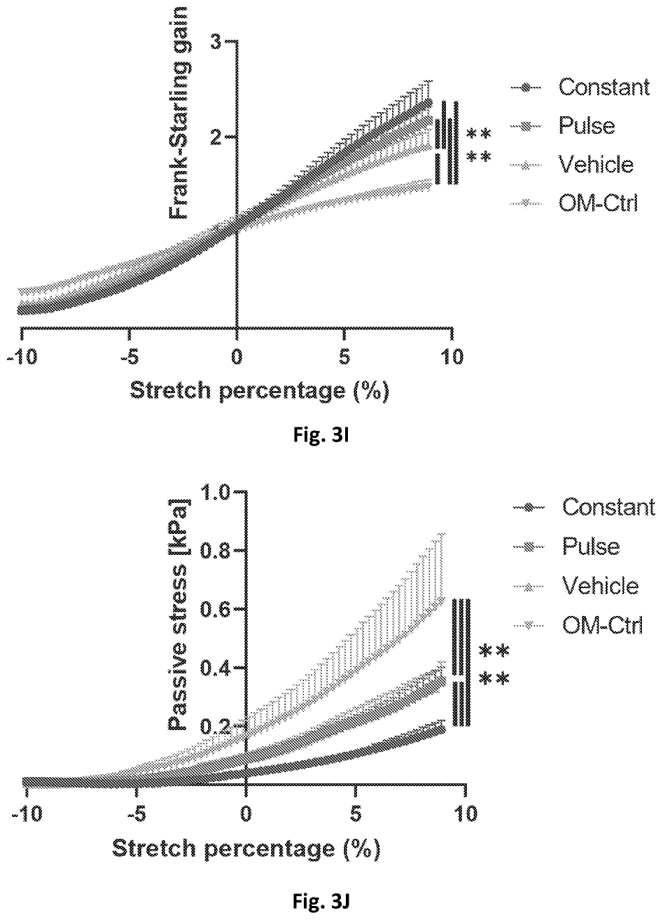

To explore potential benefits of an intermittent OM dosing strategy in a heart failure context, EHTs were constructed from a cell line engineered to express a dilated cardiomyopathy (DCM)-linked mutation to α-tropomyosin (E54K) (Mirza et al. J. Biol. Chem. 282, 13487-13497 (2007)). This experiment included four treatment groups consisting a unified set of the conditions used in the first two experiments: vehicle, constant, pulsed, and OM-Ctrl (FIG. 3A). To confirm that the 24-hr washout was sufficient to remove the acute effect of OM, a time course study was carried out for four OM-Ctrl tissues with mechanical data recorded every 30 s (FIG. 3B). Averaged curves of TTP and RT50 trendlines showed similar exponential decay behavior of OM effects, with comparable time decay constants of ˜12 minutes. The decay constants indicated that washout would be complete well before 24 hours.

Twitch force was measured in DCM EHTs from each treatment group under 1 Hz electrical pacing. (FIG. 3C). Cross-sectional areas showed similar trends as before, with constant and OM-Ctrl groups having the highest CSA values (FIG. 3D). Mean CSA of the pulse-treated tissues was lowest of all groups and was significantly lower than constant and OM-Ctrl. OM-Ctrl had the highest systolic peak force, followed by the pulse group. Constant and vehicle had identical average forces (FIG. 3E). After normalization by CSA, the pulse group peak stress became equivalent to that of the OM-Ctrl group, suggesting that even with skipping OM dosing half of the time, the pulse group still gained the same effective systolic benefit. In contrast, constant group had no normalized peak stress improvement when compared to the vehicle tissues, indicating a complete lack of sustained systolic force improvement when OM was removed after long-term constant dosing. Moreover, the pulse and constant groups showed significant TTP recovery but did not return to the vehicle control level, while OM-Ctrl predictably had the slowest TTP (FIG. 3G). As for RT50, pulse and constant groups made a complete recovery to the vehicle tissue level, whereas OM-Ctrl maintained the slowest RT50 (FIG. 3H). For length-dependent activation, OM-Ctrl had significantly lower contractility gain when stretched compared with all three other groups (FIG. 3I). In comparison, the constant and pulse both significantly improved upon the vehicle group, with no statistically significant difference between the two. Lastly, pulse and vehicle groups had near identical passive stress profiles, while the constant and OM-Ctrl group had the lowest and the highest passive stress profiles, respectively (FIG. 3J).

### Example 6

Experiments conducted with EHTs made with cells from a healthy control cell line and a mutant DCM line confirmed the hypothesis that a titrated constant OM dosing strategy does not maximize the potential chronic benefits of OM administration. Instead, significant and striking improvements were noted when EHTs were treated with an intermittent or pulsed dosing strategy. EHTs treated on alternating days with 0.5 μM OM showed consistent improvement in systolic function without exhibiting the significantly slowed twitch kinetics, blunted length-dependent activation, and diastolic function deficits observed in tissues maintained continuously in 0.5 μM OM.

For systolic function, the data show that raw contractility without further examining the normalized peak force per unit area could provide misleading conclusions. Although a continuous and constant OM dosing elevated systolic force, significantly increased cross-sectional areas of both the constant and OM-Ctrl groups resulted in reduced systolic benefits. This indicates that potential hypertrophy could have a considerable and unproductive impact on the effective contractile function, suggesting that long-term OM incubation may trigger an ineffective hypertrophy unable to produce more net contractility. On the other hand, pulse dosing produced lowered cross-sectional area and improved peak stress, indicating that more meaningful sarcomeric protein synthesis and alignment could have occurred in response to the alternating dosing regimen.

Furthermore, this in vitro study identified that pulse dosing improves Frank-Starling behavior compared to the vehicle. In contrast, constant OM dosing makes the Frank-Starling behavior worse. Next, the present study notes that pulse dosing results in a diastolic profile similar to the vehicle group in the heart failure cell line, while the constant OM dosing with washout shows reduced diastolic properties below vehicle control.

### Example 7: Effects of Enhancing Contractile Stress with Intermittent Myotrope Administration is not Limited to OM

The previously described experiments established the ability of intermittent OM administration to enhance contractile stress in the EHT system beyond the effects of constant administration.

To establish that the enhanced contractile stress in response to intermittent OM administration observed for is not limited to this specific myotrope, a follow-up study was performed to demonstrate that the same benefits can be achieved by a different cardiac myosin activator (danicamtiv).

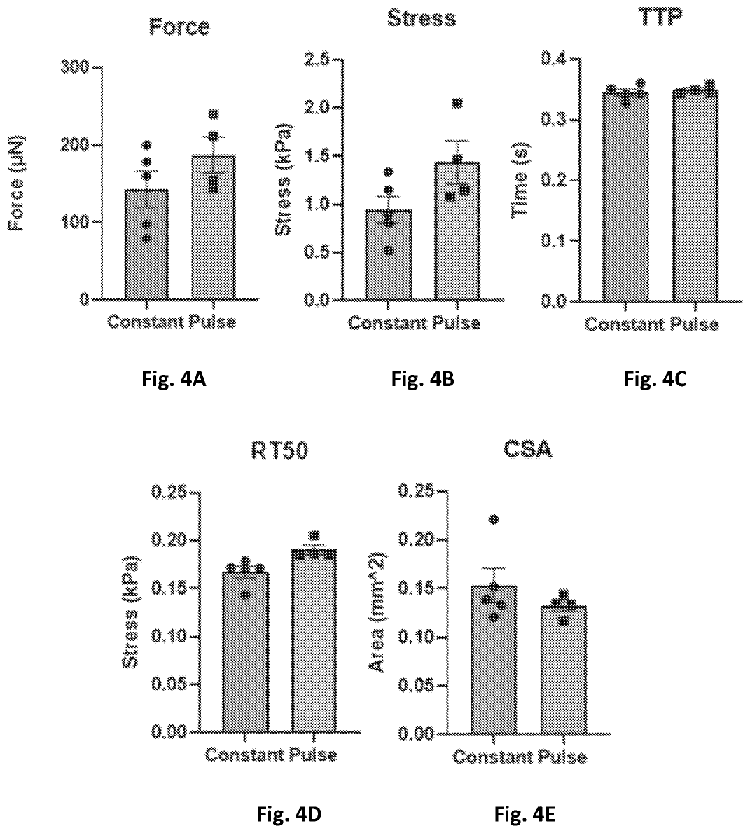

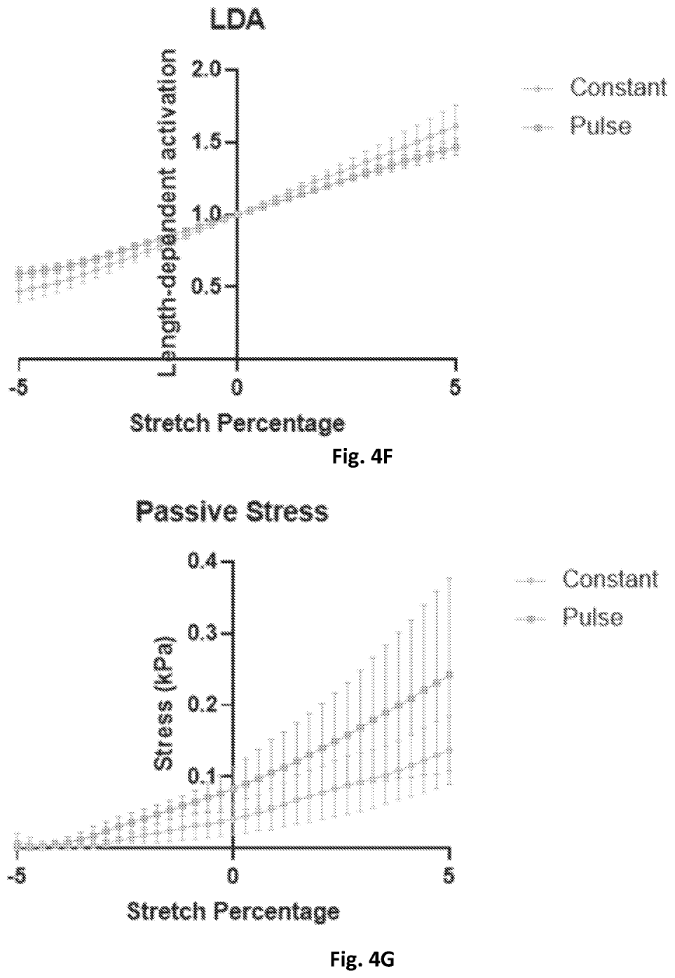

In this experiment, EHTs were formed as before from hiPSC-CMs derived from a healthy control line (GM23338, Coriell Institute). One group of EHTs was randomly selected for incubation in 1.0 μM danicamtiv over 7 days while a second group was incubated in 1.0 μM danicamtiv and then drug-free media in alternating 24-hr periods, also for 7 days (constant and pulsed groups, n=5 and n=4 respectively, FIGS. 4A-4G). The concentration of danicamtiv was selected in accordance with the EC₅₀ determined in Shen et al. (J. Am. Heart Assoc. 10, (2021)). Danicamtiv was not present during measurement of tissue performance after the 7-day incubation period, in order to expose the underlying, drug-free strength of the tissue contraction strength. Under 1 Hz electrical pacing, sample twitches showed similar kinetics but the pulse group EHTs exhibited a ˜50% greater peak systolic stress relative to the constant group (p=0.117, FIG. 4B). The pulse group also tended to have a lower cross-sectional area (CSA, FIG. 4E). Both constant and pulse groups exhibited similar time to peak twitch stress, suggesting that the differences were due to higher myofibrillar density in the pulsed group, as opposed to changes in myofilament protein isoform expression (FIG. 4C). In terms of diastolic stiffness profiles, the pulsed group showed higher passive stress and stiffness (FIG. 4G). The observations of increased peak contractile stress, decreased CSA, preserved kinetics, and increased diastolic stiffness in pulsed danicamtiv vs. constant danicamtiv was in very close agreement with the outcome of earlier experiments with omecamtiv mecarbil. It was concluded that intermittent administration of any agent that enhances myocardial contractility has the potential to enhance contractile stress beyond what is achieved from constant chronic administration of the same agent.

### Enumerated Embodiments

In some aspects, the present invention is directed to the following non-limiting embodiments:

Embodiment 1: A method of treating, ameliorating, and/or preventing heart failure in a subject in need thereof, the method comprising administering to the subject an effective amount of a myotrope using a dosing scheme having an administration-withdrawal cycle.

Embodiment 2: The method of Embodiment 1, wherein the dosing scheme comprises administering the myotrope at a frequency of less than two doses per day.

Embodiment 3: The method of any one of Embodiments 1-2, wherein the plasma concentration of the myotrope in the subject cycles between above a first threshold concentration and below a second threshold concentration during the administration, wherein the first threshold concentration is equal to or higher than the second threshold concentration.

Embodiment 4: The method of any one of Embodiments 1-3, wherein the myotrope comprises omecamtiv mecarbil (OM), APD418 (beta3-AR antagonist), danicamtiv, levosimendan, or combinations thereof.

Embodiment 5: The method of any one of Embodiments 1-4, wherein the method causes productive growth of a heart muscle in the subject.

Embodiment 6: The method of any one of Embodiments 1-5, wherein the subject is a mammal.

Embodiment 7: The method of any one of Embodiments 1-6, wherein the subject is a human.

Embodiment 8: A method of causing productive growth of a heart muscle, the method comprising intermittently contacting the heart muscle with a myotrope.

Embodiment 9: The method of Embodiment 8, wherein the heart muscle is contacted with the myotrope at a frequency of less than once per day.

Embodiment 10: The method of any one of Embodiments 8-9, wherein the concentration of the myotrope being contacted with the heart muscle cycles between above a first threshold concentration and below a second threshold concentration during the contact-withdrawal cycle, wherein the first threshold concentration is equal to or higher than the second threshold concentration.

Embodiment 11: The method of any one of Embodiments 8-10, wherein the myotrope comprises omecamtiv mecarbil (OM), APD418 (beta3-AR antagonist), danicamtiv, levosimendan, or combinations thereof.

Embodiment 12: The method of any one of Embodiments 8-11, wherein the heart muscle is in an engineered heart tissue.

Embodiment 13: The method of any one of Embodiment 8-11, wherein the heart muscle is in the heart of a subject.

Embodiment 14: The method of Embodiment 13, wherein the subject is a mammal.

Embodiment 15: The method of Embodiment 14, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating, or preventing heart failure in a subject in need thereof, the method comprising administering to the subject an effective amount of a myotrope using a dosing scheme having an administration-withdrawal cycle.

2. The method of claim 1, wherein the dosing scheme comprises administering the myotrope at a frequency of less than two doses per day.

3. The method of claim 1, wherein the plasma concentration of the myotrope in the subject cycles between above a first threshold concentration and below a second threshold concentration during the administration, wherein the first threshold concentration is equal to or higher than the second threshold concentration.

4. The method of claim 1, wherein the myotrope comprises omecamtiv mecarbil (OM), APD418 (beta3-AR antagonist), danicamtiv, levosimendan, or combinations thereof.

5. The method of claim 1, wherein the method causes productive growth of a heart muscle in the subject.

6. The method of claim 1, wherein the subject is a mammal.

7. The method of claim 1, wherein the subject is a human.

8. A method of causing productive growth of a heart muscle, the method comprising intermittently contacting the heart muscle with a myotrope.

9. The method of claim 8, wherein the heart muscle is contacted with the myotrope at a frequency of less than once per day.

10. The method of claim 8, wherein the concentration of the myotrope being contacted with the heart muscle cycles between above a first threshold concentration and below a second threshold concentration during the contact-withdrawal cycle, wherein the first threshold concentration is equal to or higher than the second threshold concentration.

11. The method of claim 8, wherein the myotrope comprises omecamtiv mecarbil (OM), APD418 (beta3-AR antagonist), danicamtiv, levosimendan, or combinations thereof.

12. The method of claim 8, wherein the heart muscle is in an engineered heart tissue.

13. The method of claim 8, wherein the heart muscle is in the heart of a subject.

14. The method of claim 13, wherein the subject is a mammal.

15. The method of claim 14, wherein the subject is a human.
